(19) United States
(12) Patent Application Publication
(10) Pub. No.: US 2023/0158342 A1
(43) Pub. Date: May 25, 2023

(54) FACE MASK

(71) Applicant: Bernard Richard Killingbeck, Fritton, Yarmouth (GB)

(72) Inventor: Bernard Richard Killingbeck, Fritton, Yarmouth (GB)

(21) Appl. No.: 17/922,532

(22) PCT Filed: Apr. 30, 2021
(86) PCT No.: PCT/GB2021/051044

§ 371 Date: Oct. 31, 2022

(30) Foreign Application Priority Data
May 1, 2020 (GB) 2007111.4

(51) Int. Cl. A62B 18/02; A62B 18/10; A62B 23/02; A61L 9/014; A41D 13/11

(52) U.S. Cl. CPC A62B 18/02; A62B 18/10; A62B 23/02; A61L 9/014; A41D 13/1107; A61L 2209/14; A61L 2209/22

(57) ABSTRACT

A face mask is provided. The mask comprises a body defining an interior side and an exterior side of the face mask; a one-way inlet valve that only allows gas to pass from the exterior side of the body to the interior side; and a one-way outlet valve that only allows gas to pass from the interior side of the body to the exterior side. By providing a face mask configured in this manner, the mask is able to provide fresh air to the wearer while reducing the likelihood of contamination by pathogens or other toxic matter.

### RELATED APPLICATIONS

The present application is a national stage application under 35 U.S.C. § 371 of International Application No. PCT/GB2021/051044, filed 30 Apr. 2021, which claims priority to Great Britain Patent Application No. 2007111,4, filled 1 May 2020. The present application claims the benefit of priority to the above referenced applications, each of which are hereby incorporated by reference in their entirety.

### BACKGROUND

Face masks are commonly used to prevent airborne transmission of pathogens by covering a wearer's mouth and nose, acting as a barrier against aerosols and respiratory droplets. They are also used as protection against air pollution and particulates,

Surgical masks, respirators, and cloth masks are each examples of face masks used for such a purpose. In use, air passes one way through the mask when the wearer inhales and then in reverse hack through the mask when exhaling. When worn correctly, face masks reduce the amount of toxic matter that can pass through from the environment to the wearer (e.g. when inhaling) and that which passes through from the wearer to the environment (e.g. when exhaling), while still providing the wearer with enough oxygen to breathe comfortably.

However, existing face masks do not stop all contamination as pathogens and other toxic matter can still be transferred to a user's lungs. While some pathogens are captured on the exterior of the mask these can then incubate in the mask and multiply, thereby increasing the likelihood of transmission. Continued use of the face mask further increases the risk of incubation and transmission as a user's breathing will saturate the mask with moisture

Substitute Specification, Clean over time and bring the temperature closer to 35° C.-36° C., ideal conditions for pathogen incubation.

Therefore, there is a need for an improved face mask that addresses these problems.

### SUMMARY

According to an aspect of the invention, there is provided a face mask comprising: a body defining an interior side and an exterior side of the face mask; a one-way inlet valve that only allows gas to pass from the exterior side of the body to the interior side; and a one-way outlet valve that only allows gas to pass from the interior side of the body to the exterior side.

By providing a face tuask in this manner the invention separates the flow of gas into and out of the face mask, avoiding moisture accumulation around the entrance to the mask and reducing the likelihood of contamination.

The body of the face mask defines an interior side and an exterior side of the mask. The interior side of the mask faces the user when worn correctly, while the exterior side of the mask faces away from the user. These “interior” and “exterior” references are also used to describe other features of the mask. A first component is generally referred to as being on the interior of a second component if it is closer to the face of the user than the second feature is.

The terms “air” and “gas” are used interchangeably throughout the application and are generally intended to refer to the (mixture of) gas in the environment around the user that is inhaled or exhaled while breathing. It will be apparent to the reader that the composition of gasses may change with the surrounding environment and use of the tuask,

The face mask is configured to cover the nose and mouth of a user and may also be configured to cover the eyes of the user.

The face mask may futher comprise: an inlet filter arranged at the inlet valve, the inlet filter comprising an inlet antimicrobial element; and an outlet filter arranged at the outlet valve, the outlet filter comprising an outlet antimicrobial element.

In this way, the filters and antimicrobial elements prevent toxic matter from entering the interior of the mask through the inlet valve or from exiting the interior of the mask through the outlet valve. In addition, they also prevent pathogens incubating on the mask the valves. Optionally, an antimicrobial coating may also be applied to other areas of the face mask such as the body.

Preferably, the antimicrobial element comprises copper or a copper alloy such as brass, bronze, or cupronickel.

In some examples of the invention, the inlet antimicrobial element is the same as the outlet antimicrobial element.

The outlet filter may have a higher gas flow rate than the inlet filter. This helps to ensure the mask provides an efficient flow of gas as the user breathes, reducing the build-up of exhaled breath within the interior of the mask. Similarly, the outlet valve may have a higher gas flow rate than the inlet valve.

The face mask may further comprise an outlet chamber defined by the outlet valve and the outlet filter such that, in use, exhaled gas may pass from the interior side of the face mask into the outlet chamber before passing to the exterior side of the face mask.

The face mask may further comprise a nitrogen adsorption element. The nitrogen adsorption element is arranged near the inlet valve and/or the inlet fitter. In this way, nitrogen from the surrounding air is retained by the nitrogen adsorption element so that air which passes through to the interior of the mask has an increased proportion of oxygen.

For example, the nitrogen adsorption element may be included in the inlet filter or may be a coating on the inlet valve. Preferably, the nitrogen adsorption element is zeolite, activated carbon, or a molecule sieve.

The outlet valve may be a first outlet valve and the mask further comprises a second outlet valve configured to release nitrogen to the exterior side of the mask.

The inlet filter may comprise an inlet mesh with a first mesh size. The outlet filter may comprise an outlet mesh with a second mesh size. Optionally, the first mesh size is smaller than the second mesh size, In this way, the flow rate of gas through the outlet mesh is greater than the flow rate of gas through the inlet filter. In art example, the inlet mesh has a 41% open area while the outlet mesh has a 56.6% open area.

Optionally, the inlet valve is arranged at a first end of the body, and the outlet valve is arranged at an opposing second end of the body. Spacing apart the, valves in this manner helps to provide fresh air for a user to breathe through the mask and to prevent the build-up of toxic matter at the valves and filters.

Preferably, in use, the first end is the top of the body and the second end is the bottom of the body. In this way, gravity also contributes to the efficient one-way flow of gas through the mask during use,

The face mask may further comprise: a frame arranged around at least a portion of the perimeter of the body; and a deformable seal on the intetior side of the frame, the deformable seal being configured to form a seal with the face of a timer.

Typically, the frame is rigid and provides a fixed structure to the face mask, improving the strength of the mask and reducing the likelihood of accidental contact with the mask by a user. The frame may cover the full perimeter of the body or just a portions) depending on the design of the face mask and arrangement of other components. The deformable seal provides a comfortable and airtight seal against the headface of the user when worn correctly.

The body of the face mask may comprise a transparent screen. This allows the mask to provide full face protection for the wearer without inhibiting their vision. Optionally, the interior of the screen nxay comprise an anti-mist coating and/or antimicrobial coating. Similarly, the exterior of the screen may comprise a UV-resistant coating and/or anti-scratch coating.

The screen may be moveably C6JE nectedto the body such that, in use, the screen may be moved relative to the body without breaking a seal formed by the deformable seal. This allows a user to teremporarily remove the screen from covering their face without breaking the secure seal. This may be particularly beneficial when a user only intends to remove the screen for a short period of time such as when eating or drinking.

The face mask further comprises a plurality of attachment points for attaching binding elements. Binding elements, such as a headband or car loops, are used to hold the mask securely in place on the headiface of the user.

### BRIEF DESCRIPTION OF THE DRAWINGS

Examples of the invention will now be described with reference to the accompanying drawings, in which:

FIGS. 1A to 1D illustrates an example face mask from several perspectives;

FIG. 2 illustrates a cross-section of the example face mask; and

FIG. 3 illustrates a cross-section of another example face mask.

### DETAILED DESCRIPTION

Referring to the figures, a face mask 1 has a body 10 that defines an interior side 12 of the face mask 1 and an exterior side 14 of the face mask 1. In use, the mask 1 is worn in front of a user's face, with the interior side 12 facing the user.

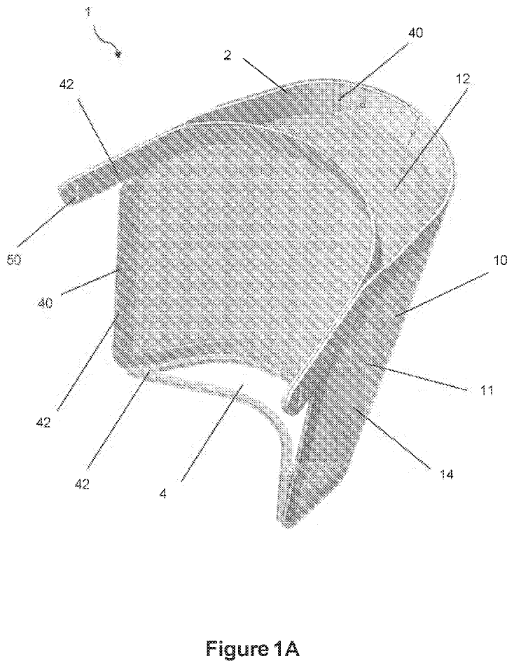

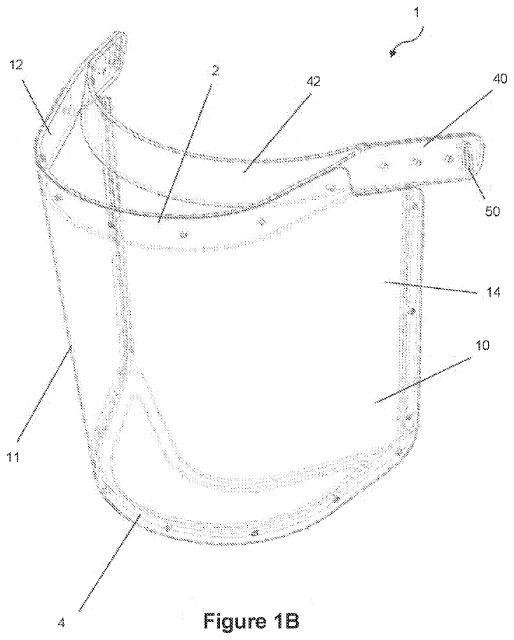

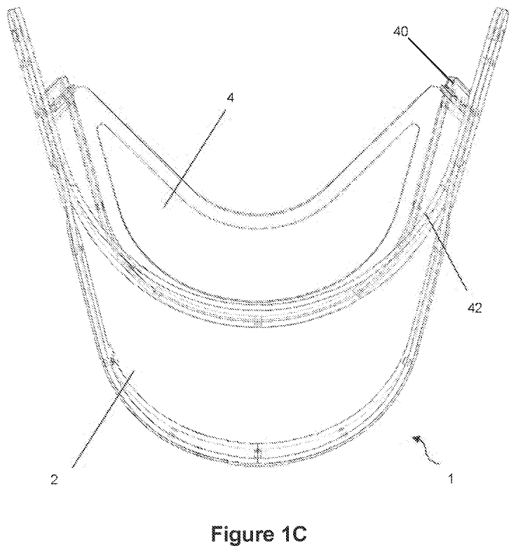

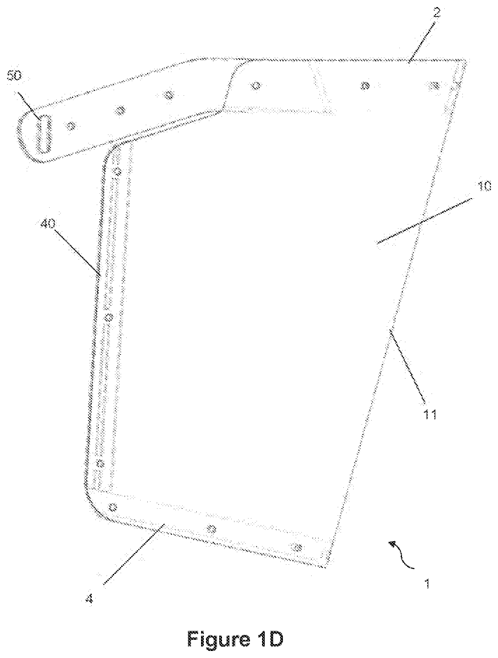

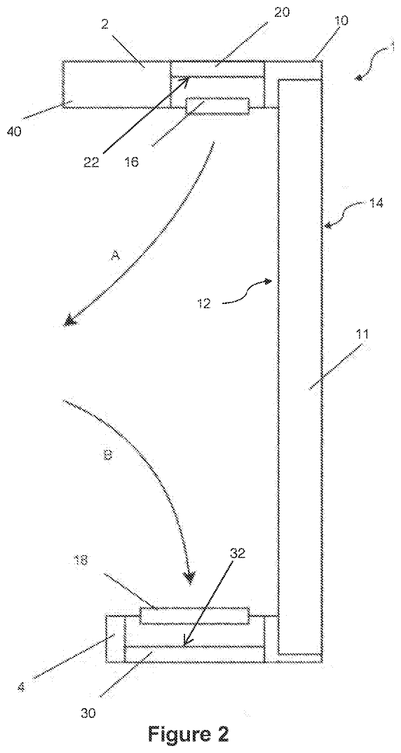

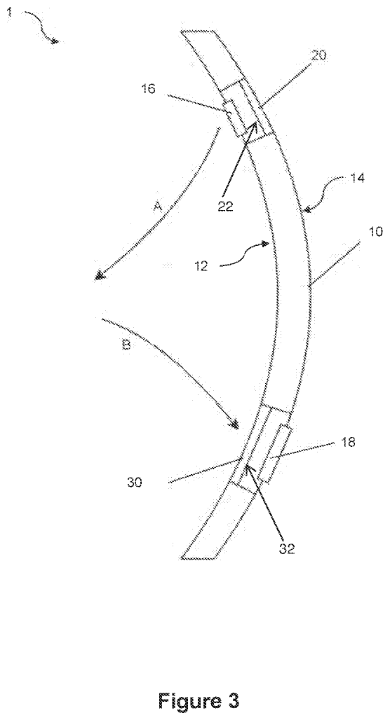

FIGS. 1A and 1B show an example face mask 1 from an angled perspective. FIG. 1C shows the same example face mask 1 from top down perspective and FIG. 1D shows the example mask 1 from a side on perspective, FIG. 2 shows a portion of the cross-section of the example face mask 1 from FIGS. 1A-D, white FIG. 3 shows a portion of the cross-section of a different example face mask 1.

The body 10 of the mask 1 does not allow gas to pass through from the exterior side 14 to the interior side 12 or vice versa. When worn correctly, the mask 1 forms an airtight seal with the face of a user and so the mask 1 comprises a plurality of valves that allow a user to breathe comfortably while wearing the mask 1. These include a one-way inlet valve 16 that only allows gas to pass from the exterior side 14 of the body to the interior side 12, and a one-way outlet valve 18 that only allows gas to pass from the interior side 12 to the exterior side 14. In use, a user may draw oxygen rich air through the inlet valve 16 into the mask 1 by breathing in and subsequently eject carbon dioxide rich air from the mask 1 through the outlet valve 18 and into the surrounding environment.

In this way, the flow of gas into and out of the mask 1 is separated, as shown in FIGS. 2 and 3 where the direction of gas flowing into the mask 1 (e.g. when the user inhales) is shown by the arrow A, and the direction of gas flowing out of the mask 1 (e.g. when the user exhales) is shown by the arrow B. As moisture is only created on the interior side 12 of the mask 1 during exhale, this defined flow direction of gas prevents moisture accumulating near the inlet valve 16 and so reduces contamination to the wearer of the mask 1

The mask 1 and valves 16, 18 are configured such that, un use, a positive air pressure is maintained on the interior side 12 of the mask 1 relative to the exterior side 14. This helps to prevent gas entering the interior side 12 of the mask if a gap between the user and the mask 1 is formed by body movement of the user. In addition, it is preferable that the outlet valvei 18 is configured to allow a higher gas flow rate than the inlet valve 16. This helps to ensure an efficient gas flow path through the mask 1 and reduces the build-up and recycling (i.e. re-breathing) of exhaled gas within the mask 1.

Preferably, the inlet valve 16 and the outlet valve 18 are spaced apart from each other. For example, the inlet valve 16 may be ananged at a first end 2 of the body 10 while the outlet valve 18 is arranged at an opposing second end 4 of the body 10. This is shown in FIGS. 1A-D and FIG. 2 where the inlet valve 16 is at the top of the body 10 and the outlet valve 18 is at the bottom of the body 10, so that gravity further contributes to the efficient one-way flow of gas through the mask 1, preventing residues of exhaled gasses such as carbon dioxide being retained within the mask 1.

As shown in the details of FIGS. 2 and 3, the face mask 1 may also include antimicrobial filters arranged nearby the valves. To he specific, an inlet filter 20 is arranged at the inlet valve 16 and an outlet filter 30 is arranged at the outlet valve 18. The inlet filter 20 prevents oxic matter Such as particulates and pathogens from passing into the interior 12 of the mask 1, thus improving the quality of air inhaled the wearer, Similarly, the outlet filler 30 stops the wearer from contaminating their environment by preventing toxic matter from exiting the mask 1.

The antimicrobial elements 22, 32 may be selected according to the anticipated use of the mask 1. In addition, the inlet antimicrobial element 22 of the inlet filter 20 may be different to the outlet antimicrobial element 32 of the outlet filter 30. For example, it is foreseeable that in some scenarios an inlet filter 20 will need to he effective at filtering a wider range of toxic matter (due to the surrounding environment) than the outlet filter 30. Preferably, the antimicrobial elements 22, 32 comprise copper or a copper alloy such as brass, bronze, or cupronickel. Copper ions (Cu2+) carry a double positive charge and react with many pathogens on contact, causing them to denature.

A valve 16, 18 and its corresponding filter 20, 30 may be arranged in either order with respect to each other. For example, FIG. 2 shows the outlet valve 18 closer to the interior side 12 and the outlet filter 30 closer to the exterior side 14, while FIG. 3 shows the outlet valve 18 and outlet filter 30 reversed. Preferably, the filter is arranged earlier in the gas flow path than its corresponding valve, as shown in FIG. 3, so as reduce contamination of the valves.

The face mask 1 may also include a nitrogen adsorption element 24 at the inlet filter 20. The nitrogen adsorption element 24 is a catalyst that retains atmospheric nitrogen from air that passes the element 24 at the inlet filter 20, thereby increasing the proportion of oxygen inhaled by a user while breathing. Preferably, the nitrogen adsorption element 24 is zeolite, activated carbon, ora molecular sieve. The mask 1 may have a second outlet valve 60 arranged at the inlet filter 20 to facilitate the release of nitrogen from the nitrogen adsorption element 24 back into the surrounding environment.

The inlet and outlet filters 20, 30 may comprise a mesh that allows gas) pass through while preventing the passage of pathogens and other toxic matter. In particular, the inlet filter 20 has an inlet mesh 21 while the outlet filter 30 includes an outlet mesh 31. in some examples, the inlet mesh 21 and the outlet mesh 31 have the same filtering properties but this is not always the case. For example, the inlet mesh 21 may have a smaller mesh size than the outlet mesh 31 so as to provide (or contribute towards) the mask 1 having a higher gas outflow rate than it does a gas inflow rate. In a specific example, the inlet mesh 21 has a 41% open area while the outlet mesh has a 56.6% open area. When the filters 20, 30 comprise a mesh, the antimicrobial elements 22, 32 are typically woven through the meshes 21, 31 to ensure that, due to the natural turbulence of gas flow and the mesh configuration, all of the gas particles contact with the antimicrobial elements 22, 32. Similarly, the nitrogen absorption element 24 may also he woven through the inlet mesh 21 and may coat the mesh 21.

Note that FIGS. 1A-D do not show several of the components such as the valves 16, 18, 60 or the filters 20, 30 but instead illustrates gaps in the body 1 where they are arranged, Optionally, these components may be removed from the mask 1 and replaced. In the example shown in FIGS. 1A-D, the valves 16, 18, 60 and filters 20, 30 would be arranged at the open regions at the first end 2 and the second end 4 of the mask 1.

In some examples of the mask 1, a valve and corresponding filter may be arranged so as to define a chamber in the body 10 between them. For example, the outlet valve 18 and outlet filter 30 may be spaced apart to define an outlet chamber 34 between them. During use, exhaled gas will pass from the interior side 12 of the face mask 1 into the outlet chamber 34 before passing to the exterior side 14 of the mask 1.

While some examples of the mask 1 (such as the cross-section shown in FIG. 3) provide a body 10 that is flexible and configured to directly fit onto a user's face, in other examples this is not the case. FIG. 1A-D show a mask 1 including a rigid frame 40 arranged around the perimeter of the body 10 to provide a more permanent structure (helping to avoid accidental contact with the mask 1) and add strength to the mask 1. The frame 49 is constructed from plastic or a lightweight metal, such as aluminium, to allow the mask 1 to be worn for extended periods of time without tiring the user. The frame 40 may be arranged around the full perimeter of the body 10 or may only occupy a portion of the perimeter.

In order to ensure the mask 1 with a frame 40 is airtight when worn correctly, the mask 1 includes a deformable seal 42 on the interior side 12 of the frame 40. Having the seal 42 comprise a deformable material such as foam means that the mask 1 can provide a comfortable and secure fit on a user without allowing gas to avoid the valves 16, 18, 60 and filters 20, 30. In some examples of the mask 1, the seal 42 may also act as an evaporator, removing moister from exhaled gas by absorption and subsequent evaporation into the atmosphere. In a similar manner, when the seal 42 is arranged nearby the nitrogen adsorption element 24 it may act as the second outlet valve 60 and release extracted nitrogen back into the surrounding atmosphere.

The body 10 may also include a transparent screen 11 so as to provide full face protection or a user without inhibiting their vision. FIG. 2 shows an example of the cross section of such a mask 1 (with the deformable seal 42 not shown). Preferably, the screen 11 is a flexible material with anti-mist and antimicrobial coatings on the interior side 12 and a UV resistant coating on the exterior side 14. In addition, some masks are configured such that the screen 11 and the body 10 or frame 40 are moveably connected to one another, for example by a hinge at the top of the mask. This allows a user to temporarily remove the screen 11 from obstructing their face without breaking the secure seal formed by the deformable seal 42.

As shown in FIGS. 1A-D, the mask 1 may also include attachment points 50. Typically, the attachment points 50 are arranged at the (periphery of the body 10 and are used for connect binding elements (not shown) such as a headband or ear ops that hold the mask 1 securely in place during use.

1. A face mask comprising:
a body defining an interior side and an exterior side of the face mask;
a one-way inlet valve that only allows gas to pass from the exterior side of the body to the interior side; and
a one-way outlet valve that only allows gas to pass from the interior side of the body to the exterior side.

2. The face mask of claim 1, further comprising:
an inlet filter arranged at the inlet valve, the inlet filter comprising an inlet antimicrobial element and
an outlet filter arranged at the outlet valve, the outlet filter comprising an outlet antimicrobial element.

3. The face mask of claim 2, wherein at least one of the inlet antimicrobial element and the outlet antimicrobial element comprises copper or a copper alloy.

4. The face mask of claim 2, wherein the inlet antimicrobial element is the same as the outlet antimicrobial element.

5. The face mask of claim 2, wherein the outlet filter has a higher gas flow rate than the inlet filter.

6. The face mask of claim 2, further comprising an outlet chamber defined by the outlet valve and the outlet filter such that, in use, exhaled gas may pass from the interior side of the face mask into the outlet chamber before passing to the exterior side of the face mask.

7. The face mask of claim 2, wherein the inlet filter comprises a nitrogen adsorption element.

8. The face mask of claim 7, wherein the outlet valve is a first outlet valve and the face mask further comprises a second outlet valve configured to release nitrogen to the exterior side of the mask.

9. The face mask of claim 2, wherein the inlet filter comprises an inlet mesh with a first mesh size.

10. The face mask of claim 9, wherein the outlet filter comprises an outlet mesh with a second mesh size.

11. The face mask of claim 10, wherein the first mesh size is smaller than the second mesh size.

12. The face mask of claim 1, wherein the inlet valve is arranged at a first end of the body, and the outlet valve is arranged at an opposing second end of the body.

13. The face mask of claim 12, wherein, in use, the first end is the top of the body and the second end is the bottom of the body.

14. The face mask of claim 1, wherein the body comprises an antimicrobial coating.

15. The face mask of claim 1, further comprising:
a frame arranged around at least a portion of the perimeter of the body: and
a deformable seal on the interior side of the frame and configured to form a seal with the face of a user.

16. The face mask of claim 15, wherein the body comprises a transparent screen.

17. The face mask of claim 16, wherein the screen is moveably connected to the body such that, in use, the screen may be moved relative to the body without breaking a seal formed by the deformable seal.

18. The face mask of claim 16, wherein the interior side of the screen comprises an anti-mist coating.

19. The face mask of claim 16, wherein the exterior side of the screen comprises a UV resistant coating.

20. The face mask of claim 1, wherein the face mask is configured to cover the eyes, nose and mouth of a user.

21. The face mask of claim 1, further comprising:
a plurality of attachment points for attaching binding elements.
